(19) United States
(12) Patent Application Publication
(10) Pub. No.: US 2026/0000614 A1
(43) Pub. Date: Jan. 1, 2026

(54) MODIFIED-RELEASE ALGINATE-BASED COMPOSITION

(71) Applicants: NUTRITION & BIOSCIENCES USA 1, LLC, Wilmington, DE (US); DANISCO (INDIA) PRIVATE LIMITED, New Dehli (IN)

(72) Inventors: Atul Ashok Kumar Lohade, New Delhi (IN); Kevin O'Donnell, Midland, MI (US); Vinay Muley, New Dehli (IN); Matthias Knarr, Bomlitz (DE); Oliver Petermann, Bomlitz (DE); Rathmaker Palarapu, New Dehli (IN)

(21) Appl. No.: 19/100,744

(22) PCT Filed: Aug. 2, 2023
(86) PCT No.: PCT/US2023/071480

§ 371 Date: Feb. 3, 2025

(30) Foreign Application Priority Data
Aug. 2, 2022 (IN) 202211044289

(51) Int. Cl. A61K 9/16; A61K 31/167

(52) U.S. Cl. CPC A61K 9/1652; A61K 9/1611; A61K 9/1617; A61K 9/1635; A61K 31/167

(57) ABSTRACT

The present invention relates to pharmaceutical raft-forming compositions suitable for modified-release formulations, methods for its preparation, as well as the use in the treatment of a medical conditions, such as a gastroesophageal reflux disease.

### FIELD OF THE INVENTION

The present invention relates to pharmaceutical raft-forming compositions suitable for modified-release formulations, methods for its preparation, as well as the use in the treatment of a medical conditions, such as a gastroesophageal reflux disease.

### BACKGROUND OF THE INVENTION

Modified-release dosage forms provide a means for less frequent dosing thereby improving patient compliance and ensuring effective and safe therapy with minimal side effects. Compared to immediate release dosage forms, modified release dosage forms can be used to prolong the action of the active ingredient after administration, and to reduce variability in the plasma concentration of a drug throughout the dosage interval, thereby eliminating or reducing sharp peaks. It has been the objective of many skilled in the art to develop such improved modified-release dosage forms due to the many advantages.

The majority of modified release dosage forms are tablets or pellets comprising a core either coated with or containing a drug. The core is then coated with a release modifying polymer within which the drug may be dispersed. The release modifying polymer disintegrates gradually, releasing the drug over time. Thus, the outer-most layer of the composition effectively slows down and thereby regulates the diffusion of the drug across the coating layer when the composition is exposed to an aqueous environment, i.e., the gastrointestinal tract. The net rate of diffusion of the drug is mainly dependent on the ability of the gastric fluid to penetrate the coating layer or matrix and on the solubility of the drug itself.

WO2020131780 relates to sustained release composition for oral administration comprises a physiologically active ingredient mixed with a methylcellulose, wherein the methylcellulose has anhydroglucose units joined by 1-4 linkages and wherein hydroxy groups of anhydroglucose units are substituted with methyl groups such that the s23/s26 is 0.27 or less, the composition further comprising a liquid diluent in a weight ratio of liquid diluent to active ingredient in the range of 0:1 to 0.85:1.

WO2020131784 relates to sustained release composition for oral administration comprises a physiologically active ingredient mixed with a methylcellulose, wherein the methylcellulose has anhydroglucose units joined by 1-4 linkages and wherein hydroxy groups of anhydroglucose units are substituted with methyl groups such that the s23/s26 is more than 0.27, and wherein the concentration of methylcellulose is from 0.1 to 10% by dry weight of the active ingredient.

WO2012128520 relates to a liquid composition for treating a gastroesophageal disease by oral administration.

There is a need for the development of easier produced, less expensive, and equally well suited modified-release composition, which by the inventors of the present invention is provided with the use of alginate rafts.

### SUMMARY OF THE INVENTION

It is an object of embodiments of the invention to provide pharmaceutical compositions suitable for a modified-release formulation, such as for oral administration, such as for

It is another object of embodiments of the invention to provide compositions suitable for use in a raft composition pharmaceutical platform.

The present invention relates in a broad aspect to raft forming pharmaceutical formulations.

Accordingly, in a first aspect the present invention relates to a two-component pharmaceutical composition suitable for a modified-release formulation consisting of compositions (I) and (II), which first raft-forming composition (I) comprises or consist of

a) an alginate in the amount of 2% (w/w) to 8% (w/w); b) a carbonate, such as an alkali metal carbonate, such as ammonium carbonate, ammonium bicarbonate, sodium carbonate or sodium bicarbonate, or calcium carbonate; c) multivalent alginate cross-linking ion, such as calcium, magnesium, aluminium, such as from a calcium carbonate in an amount of about 1.1% (w/w) to 2.2% (w/w); d) at least one pharmaceutically acceptable viscosifier in an amount of about 0.01 (w/w) to 1.0% (w/w) selected from i) a hydrocolloid, such as any one selected from the group consisting of: guar gum, pectin and its derivatives, xanthan gum, arabinoxylan, cellulose and its derivatives, chitin, xylan, beta-glucan, gum Arabic, hyaluronic acid, and gelatin; and ii) a pharmaceutically acceptable poly-acrylic acid, such as a carbomer; and e) water or other pharmaceutically acceptable vehicle; and which second composition (II) comprises or consist of a) a cellulose derivative polymer selected from the group consisting of hydroxypropyl methylcellulose (HPMC), ethylcellulose (EC), carboxymethyl cellulose (CMC), such as sodium carboxymethylcellulose, and methylcellulose (MC), hydroxypropyl cellulose (HPC); and mixtures thereof; b) an active pharmaceutical ingredient (API); and c) optionally water or any other pharmaceutically acceptable vehicle, which first (I) and second (II) composition is mixed before oral administration.

It is to be understood that components b) and c) of the first raft-forming composition (I) described herein, i.e. the carbonate and the multivalent alginate cross-linking ion respectively may be the same compound, such as wherein this carbonate source for the floating of the raft is e.g. calcium carbonate; which calcium carbonate also provides for the multivalent cross-linking ions.

In a second aspect the present invention relates to a method for the preparation of a two-component pharmaceutical composition comprising the steps of mixing each compositions (I) and (II), which first raft-forming composition (I) comprises or consist of a) an alginate in the amount of 2% (w/w) to 8% (w/w); b) a carbonate, such as an alkali metal carbonate, such as ammonium carbonate, ammonium bicarbonate, sodium carbonate or sodium bicarbonate, or calcium carbonate; c) multivalent alginate cross-linking ion, such as calcium, magnesium, aluminium, such as from a calcium carbonate in an amount of about 1.1% (w/w) to 2.2% (w/w); d) at least one pharmaceutically acceptable viscosifier in an amount of about 0.01 (w/w) to 1.0% (w/w) selected from i) a hydrocolloid, such as any one selected from the group consisting of: guar gum, pectin and its derivatives, xanthan gum, arabinoxylan, cellulose and its derivatives, chitin, xylan, beta-glucan, gum Arabic, hyaluronic acid, and gelatin; and ii) a pharmaceutically acceptable poly-acrylic acid, such as carbopol; and e) water or other pharmaceutically acceptable vehicle; and which second composition (II) comprises or consist of a) a cellulose derivative polymer selected from the group consisting of hydroxypropyl methylcellulose (HPMC), ethylcellulose (EC), carboxymethyl cellulose (CMC), such as sodium carboxymethylcellulose, and methylcellulose (MC), hydroxypropyl cellulose (HPC); and mixtures thereof; b) an active pharmaceutical ingredient (API); and c) optionally water or any other pharmaceutically acceptable vehicle.

In a third aspect the present invention relates to the use of a two-component pharmaceutical composition consisting of compositions (I) and (II), which first raft-forming composition (I) comprises or consist of a) an alginate in the amount of 2% (w/w) to 8% (w/w); b) a carbonate, such as an alkali metal carbonate, such as ammonium carbonate, ammonium bicarbonate, sodium carbonate or sodium bicarbonate, or calcium carbonate; c) multivalent alginate cross-linking ion, such as calcium, magnesium, aluminium, such as from a calcium carbonate in an amount of about 1.1% (w/w) to 2.2% (w/w); d) at least one pharmaceutically acceptable viscosifier in an amount of about 0.01 (w/w) to 1.0% (w/w) selected from i) a hydrocolloid, such as any one selected from the group consisting of: guar gum, pectin and its derivatives, xanthan gum, arabinoxylan, cellulose and its derivatives, chitin, xylan, beta-glucan, gum Arabic, hyaluronic acid, and gelatin; and ii) a pharmaceutically acceptable poly-acrylic acid, such as carbopol; and e) water or other pharmaceutically acceptable vehicle; and which second composition (II) comprises or consist of a) a cellulose derivative polymer selected from the group consisting of hydroxypropyl methylcellulose (HPMC), ethylcellulose (EC), carboxymethyl cellulose (CMC), such as sodium carboxymethylcellulose, and methylcellulose (MC), hydroxypropyl cellulose (HPC); and mixtures thereof; b) an active pharmaceutical ingredient (API); and c) optionally water or any other pharmaceutically acceptable vehicle; for use in the treatment of a medical condition for which the API is indicated.

In a further aspect the present invention relates to a method for the treatment of a medical condition for which an API is indicated comprising the administering of an effective amount to a subject in need thereof of a two-component pharmaceutical composition consisting of compositions (I) and (II), which first raft-forming composition (I) comprises or consist of a) an alginate in the amount of 2% (w/w) to 8% (w/w); b) a carbonate, such as an alkali metal carbonate, such as ammonium carbonate, ammonium bicarbonate, sodium carbonate or sodium bicarbonate, or calcium carbonate; c) multivalent alginate cross-linking ion, such as calcium, magnesium, aluminium, such as from a calcium carbonate in an amount of about 1.1% (w/w) to 2.2% (w/w); d) at least one pharmaceutically acceptable viscosifier in an amount of about 0.01 (w/w) to 1.0% (w/w) selected from i) a hydrocolloid, such as any one selected from the group consisting of: guar gum, pectin and its derivatives, xanthan gum, arabinoxylan, cellulose and its derivatives, chitin, xylan, beta-glucan, gum Arabic, hyaluronic acid, and gelatin; and ii) a pharmaceutically acceptable poly-acrylic acid, such as carbopol; and e) water or other pharmaceutically acceptable vehicle;

and which second composition (II) comprises or consist of a) a cellulose derivative polymer selected from the group consisting of hydroxypropyl methylcellulose (HPMC), ethylcellulose (EC), carboxymethyl cellulose (CMC), such as sodium carboxymethylcellulose, and methylcellulose (MC), hydroxypropyl cellulose (HPC); and mixtures thereof; b) an active pharmaceutical ingredient (API); and c) optionally water or any other pharmaceutically acceptable vehicle.

It is to be understood that the modified-release alginate compositions according to the present invention uses alginate as rafts and as the mode to make the composition floating in a gastric media, thereby providing specific drug release in the stomach, whereas the both these rafts and the cellulose derivative polymer works as a controlled-release polymer providing for a sustained release of active (API) in the gastric medium.

This provides for platform technology with a great advantage and potential to release maximum drug content in the stomach. A controlled drug release can with this inventive technology be achieved over the period of 6 to 24 hours.

The composition comprising the active pharmaceutical ingredient can be formulated in any preferred way, such as granules in a sachet, capsules, or as tablets.

This part of the formulation comprising the active pharmaceutical ingredient may be mixed with the alginate-comprising composition.

Accordingly, this concept offers a platform to achieve stomach-targeted drug release to different APIs.

### DETAILED DESCRIPTION OF THE INVENTION

As described above the present invention relates to a two-component pharmaceutical composition suitable for a modified-release formulation consisting of compositions (I) and (II), which first raft-forming composition (I) comprises or consist of
a) an alginate in the amount of 2% (w/w) to 8% (w/w); b) a carbonate, such as an alkali metal carbonate, such as ammonium carbonate, ammonium bicarbonate, sodium carbonate or sodium bicarbonate, or calcium carbonate; c) multivalent alginate cross-linking ion, such as calcium, magnesium, aluminium, such as from a calcium carbonate in an amount of about 1.1% (w/w) to 2.2% (w/w); d) at least one pharmaceutically acceptable viscosifier in an amount of about 0.01 (w/w) to 1.0% (w/w) selected from i) a hydrocolloid, such as any one selected from the group consisting of: guar gum, pectin and its derivatives, xanthan gum, arabinoxylan, cellulose and its derivatives, chitin, xylan, beta-glucan, gum Arabic, hyaluronic acid, and gelatin; and ii) a pharmaceutically acceptable poly-acrylic acid, such as a carbomer; and e) water or other pharmaceutically acceptable vehicle;
and which second composition (II) comprises or consist of a) a cellulose derivative polymer selected from the group consisting of hydroxypropyl methylcellulose (HPMC), ethylcellulose (EC), carboxymethyl cellulose (CMC), such as sodium carboxymethylcellulose, and methylcellulose (MC), hydroxypropyl cellulose (HPC); and mixtures thereof; b) an active pharmaceutical ingredient (API); and c) optionally water or any other pharmaceutically acceptable vehicle, which first (I) and second (II) composition is mixed before oral administration.

As used herein modified-release formulations refers to is a pharmaceutical formulation with a mechanism that (in contrast to immediate-release dosage) delivers a API with a delay after its administration (delayed-release dosage) or for a prolonged period of time (extended-release dosage) or to a specific target in the body (targeted-release dosage), in this case preferably in the stomach.

In some embodiments this second composition (II) is in the form of granules with a particle size of granules in the range of 200 to 2000 μm, such as in the range of 250 to 1400 μm, such as in the range of 300 to 1000 μm.

In some embodiments this second (II) composition is in the dosage form of granules, sachet, capsule, tablet, or liquid suspension.

In some embodiments the composition is in the form of a liquid dispersion after mixing of the first (I) and second (II) composition.

In some embodiments the API is a compound suitable for local therapeutic use, such as of the gastrointestinal system, such as for peptic ulcers and reflux esophagitis, such as Ranitidine, Amoxicillin, Levofloxacin, Metronidazole; low-solubility drug, such as at alkaline pH, such as Ofloxacin and Cinnarizine; compounds having a narrow absorption window like Riboflavin, Cilostazol, and Pregabalin; drugs having poor absorption from lower gastrointestinal tract like Atenolol, Lafutidine; drugs which are unstable at alkaline pH, such as Verapamil and Captopril; antidiabetic drugs, such as metformin; antihypertensive drugs, such as metoprolol hydrochloride, propranolol hydrochloride, proton pump inhibitors such as omeprazole, pantoprazole; non-steroidal anti-inflammatory analgesic drug such as paracetamol, ibuprofen; a hydrophilic compound, such as niacinamide and metoclopramide HCl; or a hydrophobic compound.

In some embodiments the cellulose derivative polymer is selected from a hydroxypropyl methylcellulose acetate succinate, a methylcellulose and an ethylcellulose.

The two-component pharmaceutical composition according to any one of claims 1-6, wherein the alginate is a salt of alginic acid, such as sodium alginate.

In some embodiments the carbonate, such as the alkali metal carbonate is sodium bicarbonate, or potassium bicarbonate or ammonium carbonate.

In some embodiments the composition further comprises a preservative, such as propyl parahydroxybenzoate and/or methyl parahydroxybenzoate; and any salt thereof.

In some embodiments the composition further comprises a flavoring and/or sweetening compound; and/or a pH modifier.

In some embodiments the alginate is present in an amount of at least 2.1% (w/w), such as at least 2.2% (w/w), such as at least 2.3% (w/w), such as at least 2.4% (w/w), such as at least 2.5% (w/w), such as at least 2.6% (w/w), such as at least 2.7% (w/w), such as at least 2.8% (w/w), such as at least 2.9% (w/w), such as at least 3.0% (w/w), such as at least 3.2% (w/w), such as at least 3.4% (w/w), such as at least 3.6% (w/w), such as at least 3.8% (w/w), such as at least 4.0% (w/w) relative to the first raft-forming composition (I).

In some embodiments the alginate is present in an amount of not more than 7.8% (w/w), such as not more than 7.6% (w/w), such as not more than 7.5% (w/w), such as not more than 7.4% (w/w), such as not more than 7.2% (w/w), such as not more than 7.0% (w/w), such as not more than 6.8% (w/w), such as not more than 6.6% (w/w), such as not more than 6.4% (w/w), such as not more than 6.2% (w/w), such as not more than 6.0% (w/w), such as not more than 5.8% (w/w), such as not more than 5.6% (w/w), such as not more than 5.4% (w/w), such as not more than 5.2% (w/w), such as not more than 5.0% (w/w), such as not more than 4.9% (w/w), such as not more than 4.8% (w/w), such as not more than 4.7% (w/w), such as not more than 4.6% (w/w), such as not more than 4.5% (w/w), such as not more than 4.4% (w/w), such as not more than 4.3% (w/w), such as not more than 4.2% (w/w), such as not more than 4.1% (w/w), such as not more than 4.0% (w/w) relative to the first raft-forming composition (I).

In some embodiments the cellulose derivative polymer is methylcellulose, such as a methylcellulose having a viscosity in the range from 15 mPa·s to 100 000 mPa·s as measured at 5 degrees C. at a concentration of 2% in water at a shear rate of 10 s⁻¹.

In some embodiments the cellulose derivative polymer is methylcellulose, such as a methylcellulose having a viscosity in the range from 150 mPa·s to 100 000 mPa·s as measured at 5 degrees C. at a concentration of 2% in water at a shear rate of 10 s⁻¹.

In some embodiments the cellulose derivative polymer is methylcellulose having s23/s26 from 0.10 to 0.24, such as from 0.14 to 0.23, wherein s23 is the molar fraction of anhydroglucose units wherein only the two hydroxy groups in the 2- and 3-positions of the anhydroglucose unit are substituted with methyl groups and wherein s26 is the molar fraction of anhydroglucose units wherein only the two hydroxy groups in the 2- and 6-positions of the anhydroglucose unit are substituted with methyl groups.

In some embodiments the ratio between the API and the cellulose derivative polymer is in the range of 1:0.001 to 1:0.1, such as in the range of 1:0.002 to 1:0.09, such as 1:0.003 to 1:0.08, such as 1:0.004 to 1:0.07, such as 1:0.005 to 1:0.06, such as 1:0.006 to 1:0.05, such as 1:0.007 to 1:0.04, such as 1:0.008 to 1:0.03, such as 1:0.009 to 1:0.02, such as around 1:0.01.

In some embodiments the cellulose derivative polymer is methylcellulose present in an amount of at least 0.1% (w/w), such as at least 0.12, 0.14, 0.16, 0.18, 0.20, 0.22, 0.24, 0.26, 0.28, 0.30, 0.32, 0.34, 0.36, 0.38, 0.40, 0.42, 0.46, 0.48, or 0.50% (w/w) relative to the second composition (II).

In some embodiments the cellulose derivative polymer is methylcellulose present in an amount of not more than 3% (w/w), such as not more than 3.0, such as not more than 2.9, 2.8, 2.6, 2.4, 2.2, 2.0, 1.9, 1.8, 1.7, 1.6, 1.5, 1.4, 1.3, 1.2, 1.1, 1.0, 0.9, 0.8, 0.7, 0.6, or 0.5% (w/w) relative to the second composition (II).

In some embodiments the cellulose derivative polymer is methylcellulose present in an amount of 0.1% (w/w)-2.0% (w/w), such as in the range of 0.2% (w/w)-1.8% (w/w), such as in the range of 0.3% (w/w)-1.7% (w/w), such as in the range of 0.4% (w/w)-1.6% (w/w), such as in the range of 0.5% (w/w)-1.5% (w/w), such as in the range of 0.6% (w/w)-1.4% (w/w), such as in the range of 0.7% (w/w)-1.3% (w/w) relative to the second composition (II).

In some embodiments the cellulose derivative polymer is hydroxypropyl methylcellulose (HPMC), such as a HPMC having a viscosity in the range of 2500 mPa·s to 120,000 mPa·s as measured at 20 degrees C. according to USP37 at c=2% in water.

In some embodiments the cellulose derivative polymer is hydroxypropyl methylcellulose (HPMC) present in an amount of at least 1% (w/w), such as at least 2, 3, 4, 5, or 6% (w/w) relative to the second composition (II).

In some embodiments the cellulose derivative polymer is hydroxypropyl methylcellulose (HPMC) present in an amount of not more than 22% (w/w), such as not more than 21, such as not more than 20, 19, 18, 17, 16, 15, or 14% (w/w) relative to the second composition (II).

In some embodiments the cellulose derivative polymer is hydroxypropyl methylcellulose (HPMC) present in an amount of 1% (w/w)-20% (w/w), such as in the range of 2% (w/w)-18% (w/w), such as in the range of 3% (w/w)-18% (w/w), such as in the range of 4% (w/w)-18% (w/w), such as in the range of 5% (w/w)-18% (w/w), such as in the range of 5% (w/w)-17% (w/w), such as in the range of 6% (w/w)-17% (w/w) relative to the second composition (II).

In some embodiments the cellulose derivative polymer is ethylcellulose, such as an ethylcellulose having a viscosity in the range of 3 mPa·s to 150 mPa·s as measured at 25 degrees C. as measured for a 5% solution measured in an Ubbelohde viscometer in the solvent 80% toluene and 20% ethanol.

In some embodiments the cellulose derivative polymer is ethylcellulose present in an amount of at least 1% (w/w), such as at least 2, 3, 4, 5, or 6% (w/w) relative to the second composition (II).

In some embodiments the cellulose derivative polymer is ethylcellulose present in an amount of not more than 22% (w/w), such as not more than 21, such as not more than 20, 19, 18, 17, 16, 15, or 14% (w/w) relative to the second composition (II).

In some embodiments the cellulose derivative polymer is ethylcellulose present in an amount of 1% (w/w)-20% (w/w), such as in the range of 2% (w/w)-18% (w/w), such as in the range of 3% (w/w)-18% (w/w), such as in the range of 4% (w/w)-18% (w/w), such as in the range of 5% (w/w)-18% (w/w), such as in the range of 5% (w/w)-17% (w/w), such as in the range of 6% (w/w)-17% (w/w) relative to the second composition (II).

In some embodiments the alkali metal carbonate, such as sodium carbonate or sodium bicarbonate is present in an amount of 1.5-5% (w/w), such as in the range of 1.6-4.8% (w/w), such as in the range of 1.7-4.6% (w/w), such as in the range of 1.8-4.4% (w/w), such as in the range of 1.9-4.2% (w/w), such as in the range of 2.0-4.0% (w/w), such as in the range of 2.1-3.8% (w/w), such as in the range of 2.2-3.6% (w/w), such as in the range of 2.3-3.4% (w/w), such as in the range of 2.4-3.2% (w/w), such as in the range of 2.5-3.0% (w/w), such as in the range of 2.0-3.5% (w/w) relative to the first raft-forming composition (I).

In some embodiments the multivalent alginate cross-linking ion, such as calcium, magnesium, aluminium, such as from a calcium carbonate is present in an amount of 0.8-4% (w/w), such as in the range of 0.9-3.9% (w/w), such as in the range of 1.0-3.8% (w/w), such as in the range of 1.1-3.7% (w/w), such as in the range of 1.2-3.6% (w/w), such as in the range of 1.2-3.5% (w/w), such as in the range of 1.2-3.4% (w/w), such as in the range of 1.2-3.3% (w/w), such as in the range of 1.2-3.2% (w/w), such as in the range of 1.2-3.0% (w/w), such as in the range of 1.2-2.8% (w/w), such as in the range of 1.2-2.6% (w/w), such as in the range of 1.2-2.4% (w/w), such as in the range of 1.2-2.2% (w/w), such as in the range of 1.2-2.0% (w/w), such as in the range of 1.3-2.0% (w/w), such as in the range of 1.3-1.8% (w/w), such as in the range of 1.4-1.8% (w/w) relative to the first raft-forming composition (I).

In some embodiments the viscosifier, such as a hydrocolloid is present in an amount of 0.05% (w/w)-1.0% (w/w), such as in the range of 0.06% (w/w)-0.8% (w/w), such as in the range of 0.07% (w/w)-0.7% (w/w), such as in the range of 0.08% (w/w)-0.6% (w/w), such as in the range of 0.09% (w/w)-0.5% (w/w), such as in the range of 0.10% (w/w)-0.40% (w/w), such as in the range of 0.12% (w/w)-0.35% (w/w).

In some embodiments the viscosifier is a pharmaceutically acceptable poly-acrylic acid, such as a carbomer, such as a carbomer Type A.

In some embodiments the first (I) and second (II) compositions are mixed to a final pharmaceutical composition.

### Alginic Acid and Salts Thereof

Alginates, derived from, inter alia, brown seaweeds are linear, unbranched bio-polymers consisting of (1-4)-linked β-D-mannuronic acid (M) and α-L-guluronic acid (G) residues. Alginates are not random copolymers but consist of blocks of similar and alternating sequences of residues, for example, MMMM, GGGG, and GMGM. In extracted form alginate absorbs water quickly. The physical properties of alginates may depend on the relative proportion of the M and G blocks. Gel formation at neutral pH requires a calcium source to provide calcium ion to interact with G-blocks. The greater the proportion of these G-blocks, the greater the gel strength.

“Alginate” is the term usually used for the salts of alginic acid, but it can also refer to all the derivatives of alginic acid and alginic acid itself; Alginate is present in the cell walls of brown algae as the calcium, magnesium and sodium salts of alginic acid. Dry, powdered, sodium alginate or potassium alginate may be obtained from an extraction process of this brown algae. The seaweed residue is then removed by filtration and the remaining alginate may then be recovered from the aqueous solution.

Another way to recover the alginate from the initial extraction solution is to add a calcium salt. This causes calcium alginate to form with a fibrous texture; it does not dissolve in water and can be separated from it. The separated calcium alginate is suspended in water and acid is added to convert it into alginic acid.

Alginates suitable for use in the practice of this invention will typically have a molecular weight such that they exhibit a viscosity in the range of 5-1,000 mPa·s. when measured at 2 wt % at 20° C. using rheometer setup with cup and bob geometry at a shear rate of 10 s⁻¹ In some embodiment, such alginates will exhibit a viscosity of between 6 and 600 mPa·s, such as between 7 and 500 mPa·s, or between 8 and 500 mPa·s when so measured. In some other embodiment, such alginates will exhibit a viscosity of between 8 and 400 mPa·s, such as between 8 and 300 mPa·s, such as between 9 and 200 mPa·s, or between 10 and 100 mPa·s when so measured. mPa·s

In some embodiments according to the present invention, a high G type alginate is used. A high G type alginate means that the alginate(s) employed in the practice of the present invention possess an average of at least 50 percent adjacent G units. In some embodiments the alginate will possess an average of at least 52 percent adjacent G units; in other embodiments such alginate will possess an average of at least 55 percent or more of adjacent G units, and in other embodiments such alginate will possess an average of at least 60, 65, or 70 percent or more of adjacent G units, as such higher the content of adjacent G units may result in improved product textures.

According to the present invention, alginate, such as alginic acid or an alginate salt is present in the amount of 2% (w/w) to 8% (w/w) based on the total weight of the final composition. In the present invention, alginate refers to any alginic acid or an alginate salt, such as sodium alginate, magnesium alginate, potassium alginate, triethanolamine alginate or propylene glycol monoglycolate.

As used herein a “multivalent alginate cross-linking ion” refers to any ion suitable for cross-link or for gel-formation of alginate. It is to be understood that this alginate gel formation may be provided by many multivalent ions including bivalent alkaline earth cations (Mg2+, Ca2+, and Sr2+), bivalent transition metal ions (Mn2+, Co2+, Cu2+, and Zn2+) as well as trivalent metal cations (Fe3+, Cr3+, Al3+, Ga3+, Sc3+, and La3+). In some specific embodiments, multivalent alginate cross-linking ion used is calcium ions or magnesium ions or aluminium ions.

### Viscosifiers

Viscosifiers or viscosifying agents are known to the person skilled in the art. Suitable viscosifiers include hydrocolloids such as any one selected from the group consisting of: guar gum, pectin and its derivatives, xanthan gum, arabinoxylan, cellulose and its derivatives, chitin, xylan, beta-glucan, gum Arabic, hyaluronic acid, and gelatin; and pharmaceutically acceptable poly-acrylic acids, such as a carbomer, such as Carbomer Type-A. For the purpose of the present invention, a viscosifyer does not include alginate or cellulose derivative polymers as specifically defined elsewhere. As used herein poly-acrylic acid (also referred to as a Carbomer) is a polymer with the formula (CH2-CHCO2H)n. The term refers to both homopolymers, copolymers, crosslinked polymers, and partially deprotonated derivatives thereof. In a water solution at neutral pH, poly-acrylic acid is an anionic polymer. Carbomer codes (910, 934, 940, 941, and 934P) are an indication of molecular weight and the specific components of the polymer. For many applications poly-acrylic acids are used in form of alkali metal or ammonium salts, e.g. sodium polyacrylate. Suitable carbomers to be used according to the present invention includes Carbopol® Polymers 71G NF, 971P NF, 974P NF, 934P NF (Lubrizol Life Science). A viscosifier may also be referred to herein as a stabilizer.

### Cellulose Derivative Polymers

Any suitable cellulose derivative polymer may be used according to the present invention. The person skilled in the art will know these suitable polymers.

Suitable film forming polymer used according to the present invention includes low-viscosity hydroxypropylcellulose (HPC), ethylcellulose (EC), methylcellulose (MC), carboxymethyl cellulose (CMC), and hydroxypropyl methylcellulose (HPMC), such as hypromellose 2910 (7-12% HP, 28-30% methoxy), hypromellose 2906 (4-7.5% HP, 27-30% methoxy), Hypromellose 2208 (4-12% HP, 19-24% methoxy), Hypromellose 1828 (23-32% HP, 16.5-20% methoxy). Commercially available carboxymethyl cellulose (CMC) include TEXTURACEL™ from IFF, Celetec™ from CPKelco, Aqualon™ from Ashland, Rheoflo® from USK Kimya A.S. and Akucell® from Nouryon (former AkzoNobel). Commercially available methylcelluloses and hydroxypropyl methylcelluloses include Japanese Pharmacopoeia METOLOSE (trademark) series and METOLOSE series for food additives from Shin-Etsu Chemical Co., Ltd., AnyCoat-C or AnyAddy (trademark) series from Lotte (former Samsung) Fine Chemicals Co., Ltd., METHOCEL (trademark) series from International Flavors & Fragrances (IFF) (Formerly DOW Chemical Company), and Benecel (trademark) series from Ashland.

Methylcellulose is one suitable cellulose derivative polymer to the present invention. Methylcellulose has anhydroglucose units joined by 1-4 linkages. Each anhydroglucose unit contains hydroxyl groups at the 2, 3, and 6 positions. Partial or complete substitution of these hydroxyls with methoxyl groups creates methylcellulose. For example, treatment of cellulosic fibers with caustic solution, followed by a methylating agent, yields cellulose ethers substituted with one or more methoxyl groups. If not further substituted with other alkyls, this cellulose ether is known as methylcellulose. Methylcellulose is characterized by the weight percent of methoxyl groups. By convention, the weight percent is an average weight percentage based on the total weight of the cellulose repeat unit, including all substituents. The content of the methoxyl group is reported based on the mass of the methoxyl group (i.e., —OCH3). The determination of the % methoxyl in methylcellulose (MC) polymer is carried out according to the United States Pharmacopeia (USP 37, “Methylcellulose”, pages 3776-3778). The % methoxyl can be converted into degree of substitution (DS) for methyl substituents, DS (methyl). DS (methyl), also designated as DS (methoxyl), of a methylcellulose is the average number of OH groups substituted with methyl groups per anhydroglucose unit. Preferably, methylcellulose has % methoxyl of 18% or more; more preferably 25% or more. Preferably, ingredient (b) has % methoxyl of 50% or less; more preferably 40% or less; and even more preferably 35% or less. Even more preferably, methylcellulose has a DS (methyl) of 1.55 or higher; more preferably 1.65 or higher; and most preferably 1.70 or higher. DS (methyl) is preferably 2.25 or lower; more preferably 2.20 or lower; and most preferably 2.10 or lower. In some embodiments according to the present invention a relevant characterization of methylcellulose is the quotient s23/s26. The numerals 2, 3, and 6 refer to the carbon atoms on the anyhdroglucose units, defined.

The parameter s23 is the molar fraction of anhydroglucose units wherein only the two hydroxy groups in the 2- and 3-positions of the anhydroglucose unit are substituted with methyl groups, and the parameter s26 is the molar fraction of anhydroglucose units wherein only the two hydroxy groups in the 2- and 6-positions of the anhydroglucose unit are substituted with methyl groups. For determining the s23, the term “the molar fraction of anhydroglucose units wherein only the two hydroxy groups in the 2- and 3-positions of the anhydroglucose unit are substituted with methyl groups” means that the two hydroxy groups in the 2- and 3-positions are substituted with methyl groups and the 6-positions are unsubstituted hydroxy groups. For determining the s26, the term “the molar fraction of anhydroglucose units wherein only the two hydroxy groups in the 2- and 6-positions of the anhydroglucose unit are substituted with methyl groups” means that the two hydroxy groups in the 2- and 6-positions are substituted with methyl groups and the 3-positions are unsubstituted hydroxy groups. The quotient s23/s26 is determined by dividing s23 by s26. According to the present invention in some embodiments the s23/s26 is 0.24 or less, such as 0.23 or less. Moreover, s23/s26 may be 0.10 or more, such as 0.14 or more. Methylcellulose having such s23/s26 ratio can be produced as generally described in International Patent Application, publication No. WO 2013/059064. A specific process for producing a methylcellulose having an above-mentioned s23/s26 ratio is described in WO2017192445 and commercial product with such s23/s26 ratio is Methocel™ Bind 112 from IFF.

The solution for the viscosity measurements of the sodium carboxymethylcellulose (CMC e.g. TEXTURACEL™ 20000 PA 07) are prepared by adding the appropriate amount of the CMC powder to the appropriate amount of water in order to achieve a concentration of 1% while stirring with an overhead lab stirrer at ambient temperature for at least 1 h. The viscosity is investigated with a rheometer (e.g. Anton Paar MCR 501) with a cup and bob geometry (e.g. CC-27) at 20 degrees C. The viscosity is in the range from 10 mPa·s to 15 000 mPa·s; in some embodiment, will exhibit a viscosity of between 15 and 12 000 mPa·s, such as between 20 and 11 000 mPa·s, or between 25 and 10 000 mPa·s when so measured.

The solution for the viscosity measurements of the methylcellulose (MC e.g. Methocel™ BIND 112) are prepared by adding the appropriate amount of the MC powder to the appropriate amount of water in order to achieve a concentration of 2% while stirring with an overhead lab stirrer at ambient temperature; afterwards the solutions are cooled to temperature below 5° C. and a stirred for at least 3 h. The viscosity is investigated with a rheometer (e.g. Anton Paar MCR 501) with a cup and bob geometry (e.g. CC-27) at 5 degrees C. The viscosity is in the range from 15 mPa·s to 100 000 mPa·s; in some embodiment, will exhibit a viscosity of between 50 and 80 000 mPa·s, such as between 100 and 75 000 mPa·s, or between 150 and 70 000 mPa·s when so measured.

The preservative may be any suitable compound known in the art of pharmaceutically acceptable compounds, such as any one selected from the group consisting of ethanol, benzethonium chloride, citric acid monohydrate, sodium salicylate, carbol, sodium benzoate, sodium dehydroacetate, oxyquinoline sulfate, potassium sorbate, benzalkonium chloride, benzeneconium chloride, Honey, 2-propanol, formalin, 1,2-hydroxypropane, human serum albumin, potassium L-glutamate, N-coconut oil fatty acid acyl-N-carboxymethyl-N-hydroxyethylethylenediamine sodium, thimerosal, boric acid, taurine, Sodium edetate, N-hexadecylviridinium chloride, 4-chloro-3-methylphenol, m-cresol, cresol, phenylethanol, 1,2-benzisothiazolin-3-one, disodium sulfite, glycerol (II) sulfate, phosphoric acid, butyl glycidyl ether, dl-camphor, sodium citrate, chlorobutanol, 2-hydroxybenzoic acid, phenyl salicylate, thymol, paraform, benzyl alcohol, sodium tetraborate, L-menthol, carboxybenzene, ethyl parahydroxybenzoate, butyl parahydroxybenzoate, propyl parahydroxybenzoate, methyl parahydroxybenzoate, methyl paraoxybenzoate, eucalyptus oil, chlorhexidine gluconate, butylhydroxytoluene, sorbic acid, borneol, β-naphthol, dehydroacetic acid, Isobutyl p-oxybenzoate, Peru balsam in color, benzoin, agar, 2-mercaptobenzimidazole, isopropyl p-oxybenzoate, n-dodecyltrimethylammonium chloride Tea Tree Oil, glyceryl caprate, polyglyceryl-2 laurate, polyglyceryl-10 laurate, ethylhexyl glycerin, glyceryl caprylate, or a combination of two or more of them.

### Pharmaceutical Active Ingredient (API)

Pharmaceutical active ingredients (also referred to as “drugs”) are pharmacologically active substances used to treat humans, animals or plants. Preferably, drugs are approved by the relevant regulatory agency for treatment of conditions occurring in humans or animals. Especially preferred drugs include, e.g., antifungals, antibiotics, anti-inflammatory, antimigraine, antihistamines, analgesics, antioxidants, nicotine, antipsychotics and life-style drugs (e.g. erectile dysfunction). More than one drug may be added in the compositions of the invention.

Preferably, the drug is in its free base form if it is basic or in its acid form if it is acidic. Preferably, the drug is a “low-solubility drug”, meaning that the drug has an aqueous solubility at physiologically relevant pH (e.g., pH 1-8) of about 0.5 mg/ml or less. As used herein the aqueous solubility (mg/mL) refers to the minimum value observed in any physiologically relevant aqueous solution (e.g., those with pH values between 1 and 8) including USP simulated gastric and intestinal buffers.

Relevant categories of suitable API's may include compounds used for a local therapeutic activity like Peptic ulcer and reflux esophagitis such as Ranitidine, Amoxicillin, Levofloxacin, Metronidazole. Drugs with low solubility at alkaline pH like Ofloxacin, Cinnarizine etc., drugs having Narrow absorption window like Riboflavin, Cilostazol, Pregabalin, drugs having Poor absorption from lower GIT like Atenolol, Lafutidine. Drugs which are unstable at alkaline pH Verapamil, Captopril. Antidiabetic drugs such as metformin, antihypertensive drugs such as metoprolol hydrochloride, propranolol hydrochloride proton pump inhibitors such as omeprazole, pantoprazole etc., a non-steroidal anti-inflammatory analgesic drug, such as paracetamol, ibuprofen etc.

### EXPERIMENTAL SECTION

Finished product formulation involves two major steps Preparation of alginate liquid followed by preparation of paracetamol modified-release formulation.

### Example 1: Paracetamol Granules 500 mg and Liquid Raft Composition

### Equipment Used to Manufacture the Formulation

1. Overhead stirrer
2. Weighing balance

Paracetamol Granules 500 mg with Raft composition

| | | | | | Combined Quantity | |
|---|---|---|---|---|---|---|
| | | | Individual Composition | | Part I and II | |
| | | | Quantity | Quantity | Combined | Only |
| Sr. | | | in gm | (% w/w) | Quantity | Solids |
| No | Ingredients | Function | (Each Part) | (Each Part) | (% w/w) | (% w/w) |

Part I Raft Composition

| | | | | | | |
|---|---|---|---|---|---|---|
| 1 | Sodium alginate | Raft forming | 50.00 | 5.00 | 4.88 | 37.11 |
| | (Protanal ® LFR 5/60) | polymer | | | | |
| 2 | Sodium bicarbonate | Buoyancy | 26.70 | 2.67 | 2.60 | 19.81 |
| | | agent | | | | |
| 3 | Calcium carbonate | Cross linking | 16.00 | 1.60 | 1.56 | 11.87 |
| | | agent | | | | |
| 4 | Sodium Hydroxide | pH modifier | 3.00 | 0.30 | 0.29 | 2.23 |
| 5 | Carbopol 971P | Viscosifier | 6.50 | 0.65 | 0.63 | 4.82 |
| 6 | Methyl (E218) | Preservative | 4.00 | 0.40 | 0.39 | 2.97 |
| | parahydroxybenzoate | | | | | |
| | (or sodium salt) | | | | | |
| 7 | Propyl (E216) | Preservative | 0.60 | 0.06 | 0.06 | 0.45 |
| | parahydroxybenzoate | | | | | |
| 8 | Sodium saccharin | Sweetener | 2.70 | 0.27 | 0.26 | 2.00 |
| 9 | DI water QS (In ml) | Vehicle | 890.50 | 89.05 | 86.86 | NA |

Part II Granule Composition

| | | | | | | |
|---|---|---|---|---|---|---|
| 10 | Paracetamol | Active | 25.00 | 99.01 | 2.44 | 18.55 |
| 11 | METHOCEL ™ | Control | 0.25 | 0.99 | 0.02 | 0.19 |
| | Bind 112 | release | | | | |
| | | Polymer | | | | |
| 12 | Purified Water | Vehicle | q.s. | Not | Not | NA |
| | | | | applicable | applicable | |

| | | | |
|---|---|---|---|
| Total Both parts | 1025.25 | 100.00 | NA |
| Total Solids (both parts) | 134.75 | | 100.00 |

### Manufacturing Process

### A) Liquid Alginate Preparation Process:

1. Add and dissolve sodium bicarbonate, calcium carbonate, and sodium saccharin in purified water. To this, add Sodium alginate and stir the mixture for 30 min.
2. Add and dissolve Carbopol into purified water under stirring. Neutralize the polymer with sodium hydroxide dissolved into water.
3. Add step 2 into step 1 and mix the dispersion well.
4. Add and dissolve methyl-paraben and propyl-paraben in hot water. Cool the solution.
5. Add step 4 to step 3 and mix well under stirring. Stir the mixture for 30 min.

### B) Paracetamol Modified-Release Formulation

Preparation of 2% METHOCEL Bind 112 gel Add METHOCEL™ Bind 112 to purified water (temperature 20-25° C.) at room temperature while stirring with an overhead lab stirrer at 750 rpm. Cool the solution to about 1.5° C. After the temperature of 1.5° C. was reached, stir the solution for 180 min at 750 rpm.

### Preparation of Paracetamol Modified-Release Granules

To obtain the paste, Paracetamol (25 gm) was mixed with METHOCEL Bind 112 solution (12.5 gm). The paste was dried at 50° C. and the mass was passed through the #18 mesh ASTM sieve to obtain the granules. The granules retained on the #24 ASTM sieve were used for dissolution studies.

### Dissolution Studies:

### Dissolution Conditions:

Media: 900 mL of 0.1 N Hydrochloric acid (pH 1.2)
Apparatus: USP type II Paddle
RPM: 50
Temperature: 37° C.

Media was added to the dissolution bowl and allowed to attain a temperature of 37° C. Paracetamol granules (equivalent to 500 mg of Paracetamol) were added to 67 mL of liquid alginate solution and mixed for 5 minutes. The mixture was added to the dissolution bowl. The aliquot was withdrawn at different time points to analyze the drug release.

### Example 2: High Dose API

Paracetamol Granule 1000 mg with Raft composition

| | | | | | Combined Quantity | |
|---|---|---|---|---|---|---|
| | | | Individual Composition | | Part I and II | |
| | | | Quantity | Quantity | Combined | Only |
| Sr. | | | in gm | (% w/w) | Quantity | Solids |
| No | Ingredients | Function | (Each Part) | (Each Part) | (% w/w) | (% w/w) |

Part I Raft Composition

| | | | | | | |
|---|---|---|---|---|---|---|
| 1 | Sodium alginate | Raft forming | 50.00 | 5.00 | 4.76 | 31.25 |
| | (Protanal ® LFR 5/60) | polymer | | | | |
| 2 | Sodium bicarbonate | Buoyancy | 26.70 | 2.67 | 2.54 | 16.69 |
| | | agent | | | | |
| 3 | Calcium carbonate | Cross linking | 16.00 | 1.60 | 1.52 | 10.00 |
| | | agent | | | | |
| 4 | Sodium Hydroxide | pH modifier | 3.00 | 0.30 | 0.29 | 1.88 |
| 5 | Carbopol 971P | Viscosifier | 6.50 | 0.65 | 0.62 | 4.06 |
| 6 | Methyl (E218) | Preservative | 4.00 | 0.40 | 0.38 | 2.50 |
| | parahydroxybenzoate | | | | | |
| | (or sodium salt) | | | | | |
| 7 | Propyl (E216) | Preservative | 0.60 | 0.06 | 0.06 | 0.38 |
| | parahydroxybenzoate | | | | | |
| 8 | Sodium saccharin | Sweetener | 2.70 | 0.27 | 0.26 | 1.69 |
| 9 | DI water QS (In ml) | Vehicle | 890.50 | 89.05 | 84.77 | NA |

Part II Granule Composition

| | | | | | | |
|---|---|---|---|---|---|---|
| 10 | Paracetamol | Active | 50.00 | 99.01 | 4.76 | 31.25 |
| 11 | METHOCEL ™ | Control | 0.50 | 0.99 | 0.05 | 0.31 |
| | Bind 112 | release | | | | |
| | | Polymer | | | | |
| 12 | Purified Water | Vehicle | q.s. | Not | Not | NA |
| | | | | applicable | applicable | |

| | | |
|---|---|---|
| Total Both parts | 1050.50 | 100.00 |
| Total Solids (both parts) | 160.00 | |

The manufacturing process and dissolution method remain the same as described in example 1 except the Paracetamol dose changes to 1000 mg

### Example 3: Paracetamol Granules 500 mg Prepared Using High Viscosity Hydroxypropyl Methyl Cellulose (METHOCEL™ K 100M Premium) and Raft Liquid

| | | | | | Combined Quantity | |
|---|---|---|---|---|---|---|
| | | | Individual Composition | | Part I and II | |
| | | | Quantity | Quantity | Combined | Only |
| Sr. | | | in gm | (% w/w) | Quantity | Solids |
| No | Ingredients | Function | (Each Part) | (Each Part) | (% w/w) | (% w/w) |

Part I Raft Composition

| | | | | | | |
|---|---|---|---|---|---|---|
| 1 | Sodium alginate | Raft forming | 50.00 | 5.00 | 4.87 | 36.63 |
| | (Protanal ® LFR 5/60) | polymer | | | | |
| 2 | Sodium bicarbonate | Buoyancy | 26.70 | 2.67 | 2.60 | 19.56 |
| | | agent | | | | |
| 3 | Calcium carbonate | Cross linking | 16.00 | 1.60 | 1.56 | 11.72 |
| | | agent | | | | |
| 4 | Sodium Hydroxide | pH modifier | 3.00 | 0.30 | 0.29 | 2.20 |
| 5 | Carbopol 971P | Viscosifier | 6.50 | 0.65 | 0.63 | 4.76 |
| 6 | Methyl (E218) | Preservative | 4.00 | 0.40 | 0.39 | 2.93 |
| | parahydroxybenzoate | | | | | |
| | (or sodium salt) | | | | | |
| 7 | Propyl (E216) | Preservative | 0.60 | 0.06 | 0.06 | 0.44 |
| | parahydroxybenzoate | | | | | |
| 8 | Sodium saccharin | Sweetener | 2.70 | 0.27 | 0.26 | 1.98 |
| 9 | DI water QS (In ml) | Vehicle | 890.50 | 89.05 | 86.71 | NA |

Part II Granule Composition

| | | | | | | |
|---|---|---|---|---|---|---|
| 10 | Paracetamol | Active | 25.00 | 92.59 | 2.43 | 18.32 |
| 11 | METHOCEL ™ | Control | 2.00 | 7.41 | 0.19 | 1.47 |
| | K100M | release | | | | |
| | Premium | Polymer | | | | |
| 12 | Purified Water | Vehicle | q.s. | Not | Not | NA |
| | | | | applicable | applicable | |

| | | | |
|---|---|---|---|
| Total Both parts | 1027.00 | 100.00 | NA |
| Total Solids (both parts) | 136.50 | | 100.00 |

The manufacturing process and dissolution method remain the same as described in example 1 except the METHOCEL™ K100M Premium gel preparation method.

METHOCEL™ K100M Premium gel preparation—Add and disperse polymer in hot purified water. Stir the dispersion for 60 min at room temperature.

### Example 4 to 6: Paracetamol Granules 500 mg Prepared Using Different Cellulose Chemistry and Raft Liquid

| | | | | | Combined Quantity | |
|---|---|---|---|---|---|---|
| | | | Individual Composition | | Part I and II | |
| | | | Quantity | Quantity | Combined | Only |
| Sr. | | | in gm | (% w/w) | Quantity | Solids |
| No | Ingredients | Function | (Each Part) | (Each Part) | (% w/w) | (% w/w) |

Part I Raft Composition

| | | | | | | |
|---|---|---|---|---|---|---|
| 1 | Sodium alginate | Raft forming | 50.00 | 5.00 | 4.86 | 36.17 |
| | (Protanal ® LFR 5/60) | polymer | | | | |
| 2 | Sodium bicarbonate | Buoyancy | 26.70 | 2.67 | 2.60 | 19.31 |
| | | agent | | | | |
| 3 | Calcium carbonate | Cross linking | 16.00 | 1.60 | 1.56 | 11.57 |
| | | agent | | | | |
| 4 | Sodium Hydroxide | pH modifier | 3.00 | 0.30 | 0.29 | 2.17 |
| 5 | Carbopol 971P | Viscosifier | 6.50 | 0.65 | 0.63 | 4.70 |
| 6 | Methyl (E218) | Preservative | 4.00 | 0.40 | 0.39 | 2.89 |
| | parahydroxybenzoate | | | | | |
| | (or sodium salt) | | | | | |
| 7 | Propyl (E216) | Preservative | 0.60 | 0.06 | 0.06 | 0.43 |
| | parahydroxybenzoate | | | | | |
| 8 | Sodium saccharin | Sweetener | 2.70 | 0.27 | 0.26 | 1.95 |
| 9 | DI water QS (In ml) | Vehicle | 890.50 | 89.05 | 86.56 | NA |

Part II Granule Composition

| | | | | | | |
|---|---|---|---|---|---|---|
| 10 | Paracetamol | Active | 25.00 | 86.96 | 2.43 | 18.08 |
| 11 | METHOCEL ™ K4M | Control | 3.75 | 13.04 | 0.36 | 2.71 |
| | Premium/ | release | | | | |
| | METHOCEL ™ E4M | Polymer | | | | |
| | Premium/ETHOCEL ® | | | | | |
| | Standard 100 | | | | | |
| | Premium | | | | | |
| 12 | Purified Water | Vehicle | q.s. | Not | Not | NA |
| | | | | applicable | applicable | |

| | | |
|---|---|---|
| Total Both parts | 1028.75 | 100.00 |
| Total Solids (both parts) | 138.25 | |

### Example 4—METHOCEL™ K4M Premium, Example 5: METHOCEL™ E4M Premium, Example 6: ETHOCEL® Standard 100 Premium

The manufacturing process and dissolution method remain the same as described in example 1 except for the polymer gel preparation method.

Polymer gel preparation for METHOCEL™—Add and disperse polymer in hot purified water. Stir the solution for 60 min at room temperature.

Polymer gel preparation for ETHOCEL™—Add and disperse polymer in purified water slowly. Stir the solution for 60 min at room temperature.

### Example 7: Ibuprofen Granules and Altered Raft Liquid (Reduced-Sodium Alginate Concentration and Xanthan Gum as a Stabilizer)

| | | | | | Combined Quantity | |
|---|---|---|---|---|---|---|
| | | | Individual Composition | | Part I and II | |
| | | | Quantity | Quantity | Combined | Only |
| Sr. | | | in gm | (% w/w) | Quantity | Solids |
| No | Ingredients | Function | (Each Part) | (Each Part) | (% w/w) | (% w/w) |

Part I Raft Composition

| | | | | | | |
|---|---|---|---|---|---|---|
| 1 | Sodium alginate | Raft forming | 30.00 | 3.00 | 2.99 | 33.58 |
| | (Protanal ® LFR 5/60) | polymer | | | | |
| 2 | Sodium bicarbonate | Buoyancy | 26.70 | 2.67 | 2.66 | 29.89 |
| | | agent | | | | |
| 3 | Calcium carbonate | Cross linking | 16.00 | 1.60 | 1.60 | 17.91 |
| | | agent | | | | |
| 4 | Sodium | Viscosifier | 5.00 | 0.50 | 0.50 | 5.60 |
| | carboxymethylcellulose | | | | | |
| | (TEXTURACEL ™ | | | | | |
| | 20000 PA 07) | | | | | |
| 5 | Xanthan gum | Viscosifier | 1.30 | 0.13 | 0.13 | 1.46 |
| | (GRINDSTED ® | | | | | |
| | Xanthan 80 PRM Plus) | | | | | |
| 6 | Methyl (E218) | Preservative | 4.00 | 0.40 | 0.40 | 4.48 |
| | parahydroxybenzoate | | | | | |
| | (or sodium salt) | | | | | |
| 7 | Propyl (E216) | Preservative | 0.60 | 0.06 | 0.06 | 0.67 |
| | parahydroxybenzoate | | | | | |
| 8 | Sodium saccharin | Sweetener | 2.70 | 0.27 | 0.27 | 3.02 |
| 9 | DI water QS (In ml) | Vehicle | 913.70 | 91.37 | 91.09 | NA |

Part II Granule Composition

| | | | | | | |
|---|---|---|---|---|---|---|
| 10 | Ibuprofen | Active | 3.00 | 99.01 | 0.30 | 3.36 |
| 11 | METHOCEL ™ Bind 112 | Polymer | 0.03 | 0.99 | 0.00 | 0.03 |
| 12 | Purified Water | Vehicle | q.s. | Not | | NA |
| | | | | applicable | | |

| | | | |
|---|---|---|---|
| Total Both parts | 1003.03 | 100.00 | NA |
| Total Solids (both parts) | 89.33 | | 100.00 |

### Manufacturing Process

### A) Liquid Alginate Preparation Process:

1. Add and dissolve sodium bicarbonate, calcium carbonate, and sodium saccharin in purified water. To this, add Sodium alginate and stir the mixture for 30 min. Add Xanthan gum and again stir the mixture for 30 min.
2. Add and dissolve sodium carboxymethylcellulose into purified water under stirring. Stir the mixture for 30 minutes.
3. Add step 2 into step 1 and mix the dispersion well.
4. Add and dissolve methyl-paraben and propyl-paraben in hot water. Cool the solution.
5. Add step 4 to step 3 and mix well under stirring. Stir the mixture for 30 min.

For Ibuprofen granule preparation and dissolution method is referred to example 1, except instead of paracetamol API Ibuprofen is added.

### Results:

### Drug Release Observations:

| Time | Example 1 | Example 2 | Example 3 | Example 4 | Example 5 | Example 6 | Example 7 |
|---|---|---|---|---|---|---|---|
| (HR) | (% Drug release) | | | | | | |
| 0 | 0 | 0 | 0 | 0 | 0 | 0 | 0 |
| 0.5 | 19 | 14 | 12 | 16 | 23 | — | 20 |
| 1 | 22 | 21 | 26 | 25 | 33 | 20 | 22 |
| 2 | 28 | 31 | 38 | 44 | 47 | 29 | 25 |
| 4 | 43 | 46 | 65 | 66 | 68 | 45 | 31 |
| 24 | 91 | 75 | 88 | 89 | 93 | 91 | 71 |

1. A two-component pharmaceutical composition suitable for a modified-release formulation consisting of compositions (I) and (II), wherein:
the first raft-forming composition (I) comprises;
a) an alginate in the amount of 2% (w/w) to 8% (w/w);
b) a carbonate;
c) multivalent alginate cross-linking ion;
d) at least one pharmaceutically acceptable viscosifier in an amount of about 0.01 (w/w) to 1.0% (w/w) selected from i) a hydrocolloid, and ii) a pharmaceutically acceptable poly-acrylic acid; and
e) water or other pharmaceutically acceptable vehicle; and
the second composition (II) comprises;
a) a cellulose derivative polymer selected from the group consisting of hydroxypropyl methylcellulose (HPMC), ethylcellulose (EC), carboxymethyl cellulose (CMC), and methylcellulose (MC), hydroxypropyl cellulose (HPC); and mixtures thereof; and
b) an active pharmaceutical ingredient (API),
which first (I) and second (II) composition are mixed before oral administration.

2. The two-component pharmaceutical composition according to claim 1, which second composition (II) is in the form of granules with a particle size of granules in the range of 200 to 2000 μm.

3-4. (canceled)

5. The two-component pharmaceutical composition according to claim 1, wherein the API is a compound suitable for local therapeutic use in the gastrointestinal system, low-solubility drug, a compound having a narrow absorption window, a drug having poor absorption from lower gastrointestinal tract a drug which is unstable at alkaline pH, an antidiabetic drug, an antihypertensive drug, a proton pump inhibitor, a non-steroidal anti-inflammatory analgesic drug, a hydrophilic compound, or a hydrophobic compound.

6. (canceled)

7. The two-component pharmaceutical composition according to claim 1, wherein the alginate is a salt of alginic acid.

8. The two-component pharmaceutical composition according to claim 1, wherein the alkali metal carbonate is sodium bicarbonate or potassium bicarbonate.

9-10. (canceled)

11. The two-component pharmaceutical composition according to claim 1, wherein the alginate is present in an amount of at least 2.1% (w/w) relative to the first raft-forming composition (I).

12. The two-component pharmaceutical composition according to claim 1, wherein the alginate is present in an amount of not more than 7.8% (w/w) relative to the first raft-forming composition (I).

13. The two-component pharmaceutical composition according to claim 1, wherein the cellulose derivative polymer is methylcellulose.

14. The two-component pharmaceutical composition according to claim 1, wherein the cellulose derivative polymer is methylcellulose having s23/s26 from 0.10 to 0.24, wherein s23 is the molar fraction of anhydroglucose units wherein only the two hydroxy groups in the 2- and 3-positions of the anhydroglucose unit are substituted with methyl groups and wherein s26 is the molar fraction of anhydroglucose units wherein only the two hydroxy groups in the 2- and 6-positions of the anhydroglucose unit are substituted with methyl groups.

15. The two-component pharmaceutical composition according to claim 13, wherein the ratio between the API and the cellulose derivative polymer is in the range of 1:0.001 to 1:0.1.

16-17. (canceled)

18. The two-component pharmaceutical composition according to claim 1, wherein the cellulose derivative polymer is methylcellulose present in an amount of 0.1% (w/w)-2.0% (w/w) relative to the second composition (II).

19. The two-component pharmaceutical composition according to claim 1, wherein the cellulose derivative polymer is hydroxypropyl methylcellulose (HPMC).

20-21. (canceled)

22. The two-component pharmaceutical composition according claim 19, wherein the cellulose derivative polymer is hydroxypropyl methylcellulose (HPMC) present in an amount of 1% (w/w)-20% (w/w) relative to the second composition (II).

23. The two-component pharmaceutical composition according to claim 1, wherein the cellulose derivative polymer is ethylcellulose.

24-25. (canceled)

26. The two-component pharmaceutical composition according to claim 23, wherein the cellulose derivative polymer is ethylcellulose present in an amount of 1% (w/w)-20% (w/w) relative to the second composition (II).

27. (canceled)

28. The two-component pharmaceutical composition according to claim 1, wherein the multivalent alginate cross-linking ion is present in an amount of 0.8-4% (w/w) relative to the first raft-forming composition (I).

29. (canceled)

30. The two-component pharmaceutical composition according to claim 1, wherein the viscosifier is a pharmaceutically acceptable poly-acrylic acid.

31. The two-component pharmaceutical composition according to claim 1, wherein the first (I) and second (II) compositions are mixed to a final pharmaceutical composition.

32. A method for the preparation of a two-component pharmaceutical composition comprising the steps of mixing each compositions (I) and (II), wherein:
the first raft-forming composition (I) comprises;
a) an alginate in the amount of 2% (w/w) to 8% (w/w);
b) a carbonate;
c) multivalent alginate cross-linking ion;
d) at least one pharmaceutically acceptable viscosifier in an amount of about 0.01 (w/w) to 1.0% (w/w) selected from i) a hydrocolloid, and ii) a pharmaceutically acceptable poly-acrylic acid; and
e) water or other pharmaceutically acceptable vehicle; and
the second composition (II) comprises:
a) a cellulose derivative polymer selected from the group consisting of hydroxypropyl methylcellulose (HPMC), ethylcellulose (EC), carboxymethyl cellulose (CMC), and methylcellulose (MC), hydroxypropyl cellulose (HPC); and mixtures thereof;
b) an active pharmaceutical ingredient (API).

33-34. (canceled)

35. A method for the treatment of a medical condition for which an API is indicated comprising the administering of an effective amount to a subject in need thereof of a two-component pharmaceutical composition consisting of compositions (I) and (II), wherein:
the first raft-forming composition (I) comprises:
a) an alginate in the amount of 2% (w/w) to 8% (w/w);
b) a carbonate;
c) multivalent alginate cross-linking ion;
d) at least one pharmaceutically acceptable viscosifier in an amount of about 0.01 (w/w) to 1.0% (w/w) selected from i) a hydrocolloid, and ii) a pharmaceutically acceptable poly-acrylic acid; and
e) water or other pharmaceutically acceptable vehicle; and
the second composition (II) comprises:
a) a cellulose derivative polymer selected from the group consisting of hydroxypropyl methylcellulose (HPMC), ethylcellulose (EC), carboxymethyl cellulose (CMC), and methylcellulose (MC), hydroxypropyl cellulose (HPC); and mixtures thereof; and
b) an active pharmaceutical ingredient (API).
